(19) United States
(12) Patent Application Publication
(10) Pub. No.: US 2010/0028937 A1
(43) Pub. Date: Feb. 4, 2010

(54) TEST STRIP FOR DETECTING GASTRIC PROBLEMS AND DETECTING METHOD THEREOF

(76) Inventors: Chia-Chen Liu, Taipei City (TW); Pei-Jiun Lai, Taipei City (TW); Tiao-Narn Chen, Taipei City (TW)

Correspondence Address: SINORICA, LLC, 2275 Research Blvd., Suite 500, ROCKVILLE, MD, 20850, US

(21) Appl. No.: 12/575,547

(22) Filed: Oct. 8, 2009

(30) Foreign Application Priority Data
Feb. 12, 2008 (CN) 200810183341.1

(51) Int. Cl. C12Q 1/02; C12M 1/00

(52) U.S. Cl. 435/29; 435/287.8

(57) ABSTRACT

The present invention provides a test strip and a detecting method thereof. The test strip comprises a first sheet, a second sheet a first filter and a second filter. The first sheet comprises a bulge portion deposited substantially in the center thereof. The second sheet comprises a depression in alignment with the bulge portion to form a sealing space. The first filter is disk-shaped and deposited in the sealing space. The first filter is used to receive a biological sample. The second filter is ring shaped and disposed in the sealing space and under the first filter to receive the biological sample coming from the first filter. The first filter comprises sodium azide, sodium dihydrogen phosphate, and urea, and the second filter comprises sodium azide, phenol red, and urea.

### BACKGROUND OF THE INVENTION

1. Technical Field

The present invention provides a test strip and a test method, more particularly a test strip and a test method to detect Helicobacter pylori.

2. Description of Related Art

In 1982, two Australian doctors, Dr. Barry J. Marshall and Dr. Robin Warren, found a pathogen, named as Helicobacter pylori later, is the pathogen causing gastric ulcer and duodenal ulcer. Helicobacter pylori may infect a host and burrow into the mucus of the stomach and therefore diminishes the protection provided by the mucus and results in the development of the gastric or duodenal ulcer.

One of the traditional methods to detect the infection of Helicobacter pylori is by the urea breath test. Patients may drink ¹³C- or ¹⁴C-labeled urea half hour before the breath test. If the patients are infected by Helicobacter pylori, the ¹³C- or ¹⁴C-labelled urea will be metabolized by the urease produced by Helicobacter pylori to ammonium cation and isotope-labeled carbon dioxide. The isotope labeled carbon dioxide can be detected in the breath of the infected patients.

However, the instrument used in the breath test is expensive and the result usually has to be double checked to rule out the false results by the microbial culture of the gastric biopsy of the patient. Nevertheless, the microbial culture of Helicobacter pylori is usually time consuming and tends to fail.

When using urea to detect the infection of Helicobacter pylori, the detecting result can be also determined by the produced ammonium cation instead of the carbon dioxide. Because the ammonium cation is weak basic in an aqueous solution, another traditional method to detect Helicobacter pylori is to apply the biological sample to a solution containing urea and phenol red, which is a pH indicator may change color in a basic environment. The color of the solution may change if the sample has Helicobacter pylori. However, the aqueous solution is not suitable for a long-term preservation in room temperature. Therefore, another detecting method is a half-wet method, for the reaction materials are added into a gel and the detecting method is carried out in the gel. Nevertheless, the reaction time of such half-wet method usually requires several hours or even up to one day. And users will have difficulty to determine the result if the change of color is minor. Such drawback may cause a false negative result.

### BRIEF SUMMARY OF THE INVENTION

In order to overcome the above mentioned shortcomings, the present invention provides a test strip comprising a first sheet, a second sheet, a first filter and a second filter. The first sheet comprises a bulge portion deposited substantially in the center thereof. The second sheet comprises a depression in alignment with the bulge portion to form a sealing space. The first filter is disk-shaped and deposited in the sealing space. The first filter is used to receive a biological sample. The second filter is ring shaped and disposed in the sealing space and under the first filter to receive the biological sample coming from the first filter. The first filter comprises sodium azide, sodium dihydrogen phosphate, and urea, and the second filter comprises sodium azide, phenol red, and urea.

Therefore, the primary object of the present invention is to provide a test strip to detect Helicobacter pylori in a dry test.

Another object of the present invention is to provide a test strip to detect Helicobacter pylori in a calorimetric approach.

Another object of the present invention is to provide a test strip to detect Helicobacter pylori rapidly.

The present invention further provides a detecting method comprising the following steps:

(1) Providing a test strip comprising a first sheet, a second sheet, a first filter and a second filter. The first sheet has a bulge portion disposed substantially in the center thereof. The second sheet has a depression in alignment with said bulge portion to form a sealing space. The first filter is disk-shaped and comprises sodium azide, sodium dihydrogen phosphate and urea, and the second filter is ring-shaped and comprises sodium azide, phenol red and urea. The second filter being disposed in said sealing space under said first filter.

(b) Providing a biological sample.

(c) Peeling the first sheet and applying the biological sample on the first filter. The urease in biological sample will operationally seep through the first filter to the second filter.

(d) Sealing the test strip. The bulge portion of the first sheet will squeeze the first filter and the second filter in the sealing space to achieve a better sealing effect.

(e) Comparing the difference between the colors of the first filter and the second filter to determine whether the sample has Helicobacter pylori or not.

Another object of the present invention is to provide a detection method to use a dry test strip to detect Helicobacter pylori.

Another object of the present invention is to provide a detection method to detect Helicobacter pylori in a calorimetric approach.

Another object of the present invention is to provide a detection method to detect Helicobacter pylori rapidly.

### BRIEF DESCRIPTION OF THE SEVERAL VIEWS OF THE DRAWINGS

The invention as well as a preferred mode of use, further objects and advantages thereof will be best understood by referring to the following detailed description of an illustrative embodiment in conjunction with the accompanying drawings, wherein:

FIG. 1 is an explosive view of the test strip of the first preferred embodiment of the present invention,

FIG. 2 is a perspective view of the test strip of the first preferred embodiment in a sealing configuration, and

FIG. 3 is a flow chart of the second preferred embodiment of the present invention.

### DETAILED DESCRIPTION OF THE INVENTION

The present invention discloses a qualitative analyzing method for detecting a specific analyte in a tissue sample in which the physical and chemical principles as well as solution application techniques involved are well known to those skilled in the art. Therefore, a detailed description of such principles and techniques is omitted herein for brevity. Besides, the drawings referred to in the following description are not drawn to actual scale and need not be so because they are intended to demonstrate features of the present invention only schematically.

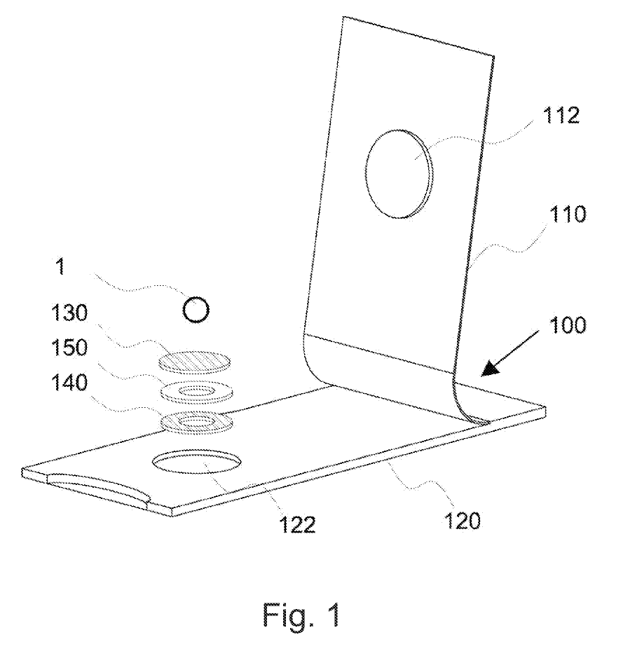

Please refer to FIG. 1 that is the explosive view of the test strip of the first preferred embodiment of the present invention. The test strip 100 is used to detect the Helicobacter pylori and comprises a first sheet 110, a second sheet 120, a first filter 130 and a second filter 140. The first sheet 110 is adhesively attached to the second sheet 120 and is generally flexible. The first sheet 110 has a bulge portion 112 that is disposed substantially the center of the first sheet 110. The second sheet 120 has a depression 122 in alignment with the bulge portion 112. The depression 122 is usually transparent. The first filter 130 and the second filter 140 are preferably has the same color, generally yellow. Users can see the color of the first filter 130 and the second filter 140 through the transparent depression 122 and then determine whether the biological sample has Helicobacter or not according to the difference between the colors of the first filter 130 and the second filter 140 after applying the biological sample.

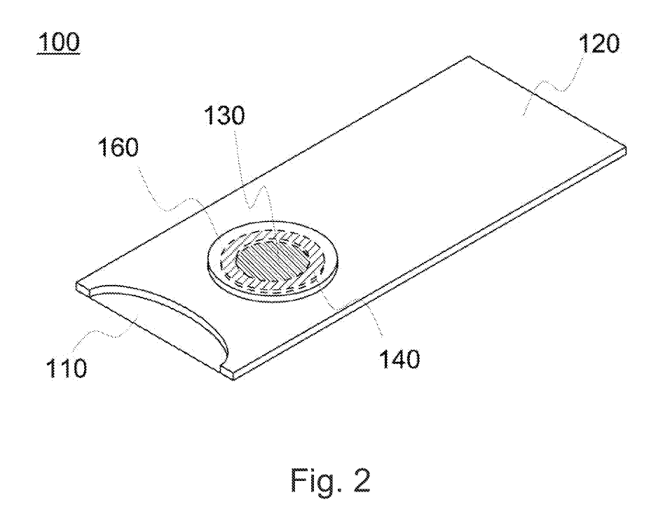

Please refer to FIG. 2 that is a perspective view of the test strip of the first preferred embodiment in a sealing configuration. The first sheet 110 is now sealing with the second sheet 120. The first filter 130 is disk shaped and used to accept the biological sample 1. The first filter 130 is disposed in the sealing space 160 defined by the bulge portion 112 and the depression 122 that is in alignment with the bulge portion 112.

The first filter 130 comprises sodium azide, sodium dihydrogen phosphate and urea. The sodium azide is used as an antibiotic agent to make the test strip 100 can be preserved longer. The sodium dihydrogen phosphate is used as a buffering agent in the testing reaction. The urea will operationally react with the urease in the biological sample and therefore is the major ingredient to detect whether the biological sample has Helicobacter pylori or not.

The second filter 140 is ring shaped and disposed in the sealing space 160 and under the first filter 130 to operationally receive the urease in the biological sample 1 seeping through the first filter 130. The second filter 140 contains sodium azide, phenol red and urea.

The function provided by the sodium azide is as previously described to be used as an antibiotic agent to make the test strip 100 to be preserved longer. The phenol red is a pH indicator. The function provided by the urea in the second filter 140 is also substantially the same as previously described to operationally react with the urease and is a major ingredient to detect whether the biological sample has Helicobacter pylori or not. If the biological sample 1 has urease, which is produced by Helicobacter pylori, the urease catalyses the urea in the first filter 130 and the second filter 140 to generate the ammonium cation. The ammonium cation is weak basic and may results in the second filter 140 with the phenol red switching from yellow to pink or red. The color of the first filter 130 will not change after applying the biological sample 1 for the first filter 130 comprises no pH indicator.

Users can determine whether the biological sample 1 has Helicobacter pylori or not from the generated difference between the colors of the first filter 130 and the second filter 140. The colors of the first filter 130 and the second filter 140 are both yellow before applying the biological sample 1. If the biological sample has Helicobacter pylori, the urease produced therefrom will make the color of the second filter 140 change and the difference between the colors of the first filter 130 and the second filter 140 is therefore achieved. The biological sample that has no Helicobacter pylori has no urease and will not make the second filter 140 changes its color.

In a preferred mode, as shown in FIG. 1, the test strip 100 can further comprises a membrane 150 disposed between the first filter 130 and the second filter 140. The membrane 150 can block the hemoglobin and other substances that may interfere the detection to seep through the first filter 130 to the second filter 140 and thus to insure the accuracy of the result of the reaction.

The concentrations of the abovementioned ingredients are preferable as below. The volumetric concentration of sodium azide preferably ranges from about 0.001% to about 0.003%. The concentration of sodium dihydrogen phosphate preferably ranges from about 6.5 mM to about 19.5 mM. The volumetric concentration of phenol red preferably ranges from about 0.025% to about 0.075%. And the volumetric concentration of urea preferably ranges from about 0.5% to about 1.5%.

The test strip 100 is a dry test strip to detect whether the biological sample 1 has Helicobacter pylori or not, and is capable of determining the result within an hour. When compared with traditional test strips, the test strip 100 provided by the present invention can detect Helicobacter pylori more rapidly. In addition, because the test strip 100 comprises two filter (the first filter 130 and the second filter 140) and only the color of the second filter 140 with phenol red will change to pink or red if the biological sample 1 has Helicobacter pylori whereas the color of the first filter 130 remained unchanged, users can simply determine whether the biological sample has Helicobacter pylori or not by the difference of the colors between the first filter 130 and the second filter 140. Therefore, the test strip 100 provide by the present invention can detect Helicobacter pylori precisely as well as rapidly.

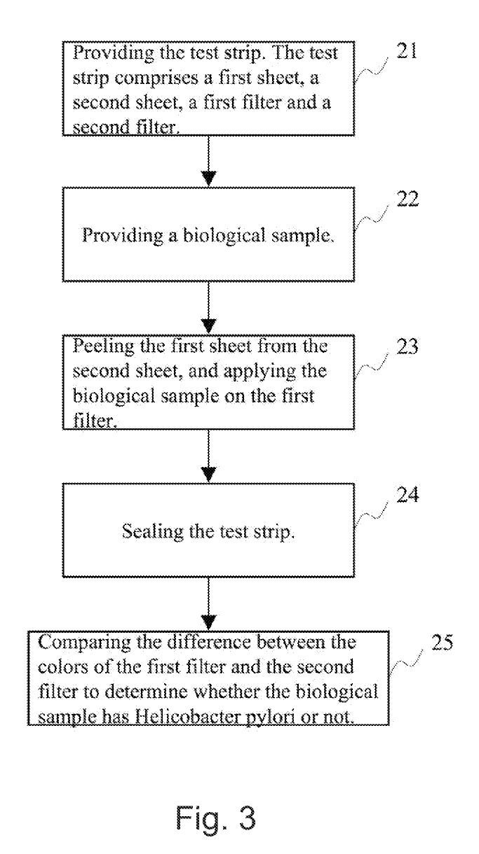

Please refer to FIG. 3. In addition to the first preferred embodiment, the present invention also provides a second preferred embodiment that is a detecting method to detect Helicobacter pylori. The detecting method comprises the following steps.

Step 21: Firstly, providing the test strip 100. The elements of the test strip 100 and the numeric labels thereof are as described in the first preferred embodiment and shown in FIG. 1 and FIG. 2. The test strip 100 comprises a first sheet 110, a second sheet 120, a first filter 130 and a second filter 140. The first sheet 110 is adhesively attached to the second sheet 120 and is generally flexible. The first sheet 110 has a bulge portion 112 that is disposed substantially the center of the first sheet 110. The second sheet 120 has a depression 122 in alignment with the bulge portion 112 to form a sealing space 160. The depression 122 is usually transparent. Users can see the color of the second filter through the transparent depression 122 and then determine whether the biological sample has Helicobacter or not according to the difference between the colors of the first filter 130 and the second filter 140.

Step 22: Providing biological sample 1. The biological sample 1 is sampled from gastrointestinal tracts by endoscopy or other surgical approaches.

Step 23: Peeling the first sheet 110 from the second sheet 120, and applying the biological sample 1 on the first filter 130. The first filter 130 is disk shaped and disposed in the sealing space 160 (FIG. 2) defined by the bulge portion 112 and the depression 122 that is in alignment with the bulge portion 112. The first filter 130 comprises sodium azide, sodium dihydrogen phosphate and urea. The second filter 140 is ring shaped and disposed in the sealing space 160 and under the first filter 130 to operationally receive the urease in the biological sample 1 seeping through the first filter 130. The second filter 140 has sodium azide, phenol red and urea.

Step 24: Sealing the test strip 100. The bulge portion 112 may squeeze the first filter 130 and the second filter 140 to achieve a better sealing effect.

Step 25: Comparing the difference between the colors of the first filter 130 and the second filter 140 to determine whether the biological sample 1 has Helicobacter pylori or not.

If the biological sample 1 has urease, which is produced by Helicobacter pylori, the urease catalyses the urea in the first filter 130 and the second filter 140 to generate the ammonium cation. The ammonium cation is weak basic and may results in the second filter 140 with the phenol red switching from yellow to pink or red. The color of the first filter 130 will not change after applying the biological sample 1 for the first filter 130 comprises no pH indicator. The colors of the first filter 130 and the second filter 140 are both yellow before applying the biological sample 1. Therefore, the first filter 130 may be a reference for whether the color of the second filter 140 changes or not.

Users can determine whether the biological sample 1 has Helicobacter pylori or not from the generated difference between the colors of the first filter 130 and the second filter 140. If the biological sample has Helicobacter pylori, the urease produced therefrom will make the color of the second filter 140 change and the difference between the colors of the first filter 130 and the second filter 140 is therefore achieved. The biological sample that has no Helicobacter pylori has no urease and will not make the second filter 140 changes its color.

In a preferred mode, as shown in FIG. 1, the test strip 100 can further comprises a membrane 150 disposed between the first filter 130 and the second filter 140. The membrane 150 can block the hemoglobin and other substances that may interfere the detection to seep through the first filter 130 to the second filter 140 and thus to insure the correctness of the result of the reaction.

The preferred concentrations of the ingredients of the test strip 100 are as described in the first preferred embodiments and therefore omitted hereinafter.

Therefore, the detecting method mentioned above is achieved by determining the difference between the color of the two filters, which can detect Helicobacter pylori precisely as well as rapidly.

The above description is intended only to demonstrate the preferred embodiment of the present invention and not to limit the scope of the invention. Moreover, as the contents disclosed herein should be readily understood and can be implemented by those skilled in the art, all equivalent changes or modifications which do not depart from the spirit of the present invention should be encompassed by the appended claims.

1. A test strip for detecting Helicobacter pylori comprising:
a first sheet comprising a bulge portion deposited substantially in the center thereof,
a second sheet comprising a depression in alignment with said bulge portion to form a sealing space,
a first filter to receive a biological sample, said first filter comprising a disk configuration and being deposited in said sealing space,
the test strip being characterized in that:
said test strip further comprising a second filter with a ring configuration which is disposed in said sealing space and under said first filter to receive the biological sample coming from said first filter,
wherein said first filter comprises sodium azide, sodium dihydrogen phosphate, and urea, and
said second filter comprises sodium azide, phenol red, and urea.

2. The test strip of claim 1, wherein said sodium azide has a volumetric concentration ranging from about 0.001% to about 0.03%.

3. The test strip of claim 1, wherein said phenol red has volumetric concentration ranging from about 0.025% to about 0.075%.

4. The test strip of claim 1, wherein said sodium dihydrogen phosphate has a concentration ranging from about 6.5 mM to about 19.5 mM.

5. The test strip of claim 1, further comprising a membrane disposed between said first filter and said second filter.

6. The test strip of claim 5, wherein said sodium azide has a volumetric concentration ranging from about 0.001% to about 0.03%, said phenol red has volumetric concentration ranging from about 0.025% to about 0.075%, said sodium dihydrogen phosphate has a concentration ranging from about 6.5 mM to about 19.5 mM, said urea has a volumetric concentration ranging from about 0.5% to about 1%, and said phenol red has volumetric concentration ranging from about 0.025% or 0.075%.

7. A method to detect Helicobacter pylori which is characterized in comprising the following steps:
providing a test strip comprising a first sheet, a second sheet, a first filter and a second filter, wherein said first sheet has a bulge portion disposed substantially in the center thereof, said second sheet has a depression in alignment with said bulge portion to form a sealing space, said first filter is in a disk configuration and comprises sodium azide, sodium dihydrogen phosphate and urea, and said second filter is in a ring configuration and comprises sodium azide, phenol red and urea, said second filter being disposed in said sealing space under said first filter,
providing a biological sample,
peeling said first sheet and applying said biological sample on said first filter,
sealing said test strip, wherein said bulge portion squeezes said first filter and said second filter to achieve a better sealing effect, and
comparing the difference between the colors of said first filter and said second filter to determine whether said sample has Helicobacter pylori or not.

8. The method of claim 7, wherein said sodium azide has a volumetric concentration ranging from about 0.001% to about 0.03%.

9. The method of claim 7, wherein said phenol red has volumetric concentration ranging from about 0.025% to about 0.075%.

10. The method of claim 7, wherein said sodium dihydrogen phosphate has a concentration ranging from about 6.5 mM to about 19.5 mM.

11. The method of claim 7, wherein said test strip further comprises a membrane disposed between said first filter and said second filter.

12. The method of claim 11, wherein said sodium azide has a volumetric concentration ranging from about 0.001% to about 0.03%, said phenol red has volumetric concentration ranging from about 0.025% to about 0.075%, said sodium dihydrogen phosphate has a concentration ranging from about 6.5 mM to about 19.5 mM, said urea has a volumetric concentration ranging from about 0.5% to about 1%, and said phenol red has volumetric concentration ranging from about 0.025% or 0.075%.
